(19) United States
(12) Patent Application Publication
(10) Pub. No.: US 2014/0277163 A1
(43) Pub. Date: Sep. 18, 2014

(54) REINFORCEMENT SYSTEMS FOR SPINE STABILIZATION CONSTRUCTS

(71) Applicants: Ryan Kretzer, Tucson, AZ (US); Scott Kokones, Brookline, MA (US); Gregory Schulte, Minneapolis, MN (US); Jeffrey Gordon, Seattle, WA (US)

(72) Inventors: Ryan Kretzer, Tucson, AZ (US); Scott Kokones, Brookline, MA (US); Gregory Schulte, Minneapolis, MN (US); Jeffrey Gordon, Seattle, WA (US)

(21) Appl. No.: 14/215,555

(22) Filed: Mar. 17, 2014

Related U.S. Application Data
(60) Provisional application No. 61/787,763, filed on Mar. 15, 2013.

(51) Int. Cl. A61B 17/70

(52) U.S. Cl. CPC A61B 17/7049; A61B 17/7035
USPC 606/278

(57) ABSTRACT

Reinforcement systems including clamp devices and reinforcement rods to stabilize pre-existing or co-existing spine stabilization constructs.

### CROSS-REFERENCE TO RELATED APPLICATIONS

The present application claims priority to U.S. Provisional Application Ser. No. 61/787,763, filed on Mar. 15, 2013 and incorporated by reference herein.

### TECHNICAL FIELD

The present invention generally relates to reinforcement systems including clamp devices and reinforcement rods to stabilize pre-existing or co-existing spine stabilization constructs.

### BACKGROUND

Mechanical fixation of a spinal level requires attachment of a semi-rigid/rigid fixator to portions of the spinal elements (vertebral body, pedicle, lateral mass, transverse process, facet joint, lamina, spinous process). The most common forms of rigid screw-rod based fixation used in clinical practice include attachment of a bone screw to either the pedicle or lateral mass at a given vertebral level. These systems are typically used to provide rigid internal fixation for unstable spinal segments to allow bone healing and fusion.

During certain spinal procedures or in the setting of severe spinal trauma, traditional bilateral single rod fixation may not provide sufficient stability to protect the neural elements and allow bony fusion. This may occur in the setting of 3-column spinal trauma or oncological disease, or in the setting of a pedicle subtraction osteotomy (PSO), vertebral column resection (VCR), spondylectomy, multiple contiguous vertebral corpectomies, deformity (scoliosis/kyphosis), bridging a junctional segment of the spine (cervicothoracic or thoracolum bar junction), or in long-segment thoracolumbosacropelvic fixation. In this case, additional points of fixation allowing bilateral dual rod placement would be beneficial to stabilize the unstable spine and allow arthrodesis to occur.

Current connection systems in the art allow uniaxial motion, minimizing the degrees of freedom available for rod-to-rod linkage. This complicates the surgical procedure, requiring additional time for rod contouring and putting added and unnecessary stress on the spinal fixation system.

### SUMMARY

The present invention relates to reinforcement systems for pre-existing or co-existing spine stabilization constructs. Embodiments of reinforcement systems are rod-to-rod connection systems that provide, for example, polyaxial adaptability for adjacent segment fixation, linkage of sequential spinal constructs, and dual rod fixation for strengthening of unstable spinal segments. Such systems serve an unmet need in spinal surgery, decreasing procedural time and increasing patient safety in the care of complex spinal disease. Such systems also minimize the risk of screw/rod breakage by sharing stress across multiple fixation points and rod segments.

Polyaxial fasteners that are part of reinforcement systems as described below can be used to adjoin multiple spinal rods in parallel fashion. Further, such polyaxial fasteners can also allow substantially perpendicular attachment of an adjoining spinal rod in the setting of a spinal decompression procedure. In this case, the perpendicular fastener/rod construct forms a midline connecting structure for bridging to an adjacent spinous process in the setting of adjacent spinal disease. This serves an unmet need in spinal revision surgery by allowing midline posterior segmental fixation to be achieved without the placement of adjacent pedicle screws, through a minimally invasive, tissue-sparing approach.

In an embodiment, the present invention provides a reinforcement system for a spine stabilization construct. The reinforcement system comprises in an operative configuration at least two clamp devices. Each clamp device comprises a polyaxial fastener defining a recess securing a reinforcement rod therein. Each clamp device also comprises a base positioned below and engaged with the polyaxial fastener. The base comprises a groove or a hole securing a primary rod therein of the spine stabilization construct. In certain embodiments, the groove or the hole is positioned between at least two spinal screws of the spine stabilization construct. In such embodiments, the spinal screws are attached to the spine at one end and attached to a primary rod of the spine stabilization construct at another end. The reinforcement system further includes a reinforcement rod secured in the recesses of the polyaxial fasteners of the at least two clamp devices and connected to the primary rod via the at least two clamp devices. Two reinforcement systems can be used with one system located on one side of the spine and the other system located on the other side of the spine.

In another embodiment, the present invention provides a reinforcement system for a spine stabilization construct. The reinforcement system comprises in an operative configuration at least two clamp devices. Each clamp device comprises a housing comprising an aperture extending along a first axis; and a bore extending along a second axis substantially perpendicular to the first axis. The bore secures a reinforcement rod therein. The housing further includes a groove extending along a third axis substantially perpendicular to the first and second axes. The groove secures a primary rod therein. The clamp device further includes a set locking screw threadably engaged with the aperture of the housing.

The reinforcement system also includes opposing rigid plates each having an upper portion and a lower portion, the lower portions connected to a reinforcement rod and the upper portion connected to a spinous process of the spine. The system further comprises a reinforcement rod connected to the lower portions of the opposing rigid plates and the at least two clamp devices.

### BRIEF DESCRIPTION OF DRAWINGS

FIG. 1 is a perspective view of a reinforcement system including a clamp device according to an embodiment of the present invention;

FIG. 2 is an exploded view of the clamp device depicted in FIG. 1;

FIG. 3 is a schematic illustration of the spine with a spine stabilization construct attached thereto;

FIG. 4 is a schematic illustration of the spine with an embodiment of a reinforcement system including clamp devices and reinforcement rods attached to the spine stabilization construct illustrated in FIG. 3;

FIG. 5 is a partial perspective view of an embodiment of a reinforcement system of the present invention attached to a primary rod of spine stabilization system and attached to a reinforcement rod;

FIG. 6 is a side view of an embodiment of a reinforcement system in relation to spinal screws;

FIG. 7 is perspective view of a reinforcement system including a clamp device according to an alternative embodiment of the present invention;

FIG. 8 is a perspective view of a reinforcement system including a clamp device according to an alternative embodiment of the present invention;

FIG. 9 is a schematic illustration of the spine with an embodiment of a reinforcement system attached to a spine stabilization construct;

FIG. 10 is perspective view of a reinforcement system according to an alternative embodiment of the present invention;

FIG. 11 is a perspective view of a reinforcement system according to an alternative embodiment of the present invention;

FIG. 12 is a schematic illustration of the spine with a spine stabilization construct attached thereto;

FIG. 13 is a schematic illustration of the spine with an embodiment of a reinforcement system attached to the spine stabilization construct illustrated in FIG. 12;

FIG. 14 is a partial perspective view of an embodiment of a reinforcement system of the present invention attached to a reinforcement rod and a primary rod;

FIG. 15 is an exploded view of a reinforcement system including a clamp device according to an alternative embodiment of the present invention;

FIG. 16 is an exploded view of a reinforcement system including the clamp device depicted in FIG. 15 and a reinforcement rod and a primary rod;

FIG. 17 is a partial perspective view of a reinforcement system according to an embodiment of the present invention attached to a primary rod;

FIG. 18 is a cross-section of a reinforcement system according to an embodiment of the present invention;

FIG. 19 is a perspective view of a housing of a clamp device of a reinforcement system according to an embodiment of the present invention.

FIG. 20 is a partial perspective view of a reinforcement system including a clamp device and reinforcement rod attached to a primary rod according to an embodiment of the present invention; and

FIG. 21 is a partial perspective view of a reinforcement system including a clamp device and a reinforcement rod attached to a primary rod according to an embodiment of the present invention.

### DETAILED DESCRIPTION

The present invention provides various reinforcement systems for spine stabilization constructs. The disclosure herein refers to the term “substantially” with respect to certain geometric shapes, orientations and configurations. By “substantially” is meant that the shape, orientation or configuration of the described component, feature or element need not have the mathematically exact described shape, orientation or configuration, but can have a shape, orientation or configuration that is recognizable by one skilled in the art as generally or approximately having the described shape, orientation or configuration. Also, the disclosure herein refers to an “operative configuration.” This is the configuration of the system when the reinforcement system has been implanted into the patient and is attached either directly or indirectly to a spine stabilization construct. The disclosure also refers to the term “integral” or “integrally attached.” By “integral” or “integrally attached” is meant that the described components are molded as one piece during manufacturing or the described components are otherwise not separable using a normal amount of force without damaging the integrity (i.e. tearing) either component. A normal amount of force is the amount of force a user would use to remove a component meant to be separated from the other component without damaging either structure. The disclosure refers to a “primary rod” and a “reinforcement rod.” A primary rod includes a spinal rod that is part of a pre-existing or co-existing spine stabilization construct. A reinforcement rod is a rod that is directly or indirectly attached to the primary rod to reinforce the primary rod and the spine stabilization construct.

Further, as used herein with respect to a described component, feature or element, the terms “a,” “an,” and “the” include at least one or more of the described component unless otherwise indicated. In addition, It will be understood that when an element is referred to as being “on,” “attached” to, “connected” to, “coupled” with, “contacting,” etc., another element, it can be directly on, attached to, connected to, coupled with, or contacting the other element or intervening elements may also be present. In contrast, when an element is referred to as being, for example, “directly on,” “directly attached” to, “directly connected” to, “directly coupled” with, or “directly contacting” another element, there are no intervening elements present. It will also be appreciated by those of skill in the art that references to a structure or feature that is disposed “adjacent” another feature may have portions that overlap or underlie the adjacent feature.

Reinforcement systems as disclosed herein provide a clamp system for joining or connecting two orthopedic structures together. The connection may be substantially parallel but in other embodiments, the connection is substantially perpendicular. The present invention provides devices for attaching a spinal rod to another spinal rod and for attaching two spinal rods in series. During an orthopedic procedure, as is known in the art, a spinal screw is inserted into the patient's spine. At least two spinal screws are implanted on each side of the spine. On each side, a spinal rod connects the two spinal screws. An example of this type of spine stabilization construct is illustrated in FIG. 3. In other procedures, a spinal rod connects between two spinal rods on either side of the spine. This type of arrangement is used to provide rotational stability in a construct. It is generally used in the presence of a laminectomy.

Devices, as disclosed herein, can include a “drop in” groove for easy insertion of a spinal rod, a primary rod, reinforcement rod, or other linking rod. Devices, as disclosed herein, can also include components for attaching a spinal rod to another spinal rod, which can snap onto the another spinal rod and may require tightening only one screw for attachment. Systems and devices of the present invention can also provide for attaching a spinal rod to another spinal rod to accommodate attachment of skew members.

In an embodiment, the present invention provides a reinforcement system for a spine stabilization construct comprising a clamp device. The clamp device comprises a polyaxial fastener defining a recess configured to accept a portion of a reinforcement rod. The clamp device also includes a base positioned below and engaged with the polyaxial fastener in an operative configuration. The base comprises a groove or a hole configured to accept a portion of a primary rod of the spine stabilization construct. As stated above, the recess of the polyaxial fastener can provide a “drop in” slot for easy insertion of a reinforcement rod. The groove can have an arcuate configuration, such as a substantially U-shaped configuration, to allow a “snap fit” of a reinforcement rod thus requiring only tightening of one screw during the reinforcement procedure.

FIG. 4 illustrates an example of a reinforcement system with a clamp devices and primary rods and reinforcement rods. FIGS. 1 and 2 depict an example of a clamp device, particularly a polyaxial fastener and a base. It is understood that other types of polyaxial fasteners can also be used so long as the fastener is able to accept a reinforcement rod as described above.

Referring to FIGS. 1 and 2, an embodiment of the present invention provides a reinforcement system 80 for a spine stabilization construct. An example of a spine stabilization construct 139 is schematically illustrated in FIG. 3. Reinforcement system 80 comprises a clamp device 90. Clamp device 90 comprises a polyaxial fastener system 91 and a base 89. Polyaxial fastener system 91 comprises a housing 88, preferably tulip-shaped, having opposing walls 92a and 92b that are at least partially internally threaded as shown in FIG. 2. Alternatively, the housing be at least partially externally threaded. In a preferred embodiment, the height of housing 88 is less than the height of spinal screw 60 in order to reduce the overall height of the reinforcement system.

Opposing walls 92a and 92b are separated by a recess 86 configured to accept a portion of a reinforcement rod 48 as illustrated in FIG. 4. In order to lock reinforcement rod 48 in housing 88, clamp device 90 further comprises a locking set screw 81 threadably engageable via threading 83 with the at least partially internally threaded opposing walls 92a and 92b of housing 88. As stated above, recess 86 can provide a “drop in” slot for easy insertion of a reinforcement rod.

In an operative configuration, locking set screw 81 is threadably engaged with the internal threading of opposing walls 92a and 92b to secure reinforcement rod 48 in place. Again, the opposing walls of the housing can be at least partially externally threaded and a locking set screw can threadably engage the external threadings of the housing to secure a reinforcement rod in place.

Referring again to FIGS. 1 and 2, clamp device 90 can further comprises an internal fastener 84 comprising a head 55 having a rounded bottom portion 65 and a shaft 93 connected to the head 55.

Clamp device 90 further comprises a base 89 positioned below housing in an operative configuration. Base 89 comprises an aperture 87 extending along a first axis and engageable with shaft 93 of the internal fastener 84. Base 89 further includes a groove 189 or hole extending along a second axis substantially perpendicular to the first axis. The groove 189 or hole is configured to accept a portion of a primary rod 44 (illustrated in FIG. 4) of the spine stabilization construct 139. To securely clamp onto primary rod 44, internal fastener 84 is inserted into aperture 87 and urged against the outer surface of primary rod 44 to secure spinal rod 44 in place. As stated above, groove 189 can have an arcuate configuration, such as a substantially U-shaped configuration, to allow a “snap fit” of a reinforcement rod thus requiring only tightening of one screw during the reinforcement procedure.

In the embodiment depicted in FIGS. 1 and 2, groove 189 and aperture 87 of the base 89 are substantially aligned such that the first axis and second axis intersect. Further, in this embodiment, the internal fastener 84 is a set screw, shaft 93 is externally threaded, aperture 87 is internally threaded, and aperture 87 is threadably engageable with externally threaded shaft 93 of set screw (84). In an operative configuration, internal fastener 84 is threadably engaged with the internal threading of aperture 87 and is secured against primary rod 44.

Clamp device 90 thereby attaches primary rod 44 to a reinforcement rod 48 using a locking set screw 81, an internal set screw 84, a housing 88 and a base 89. The polyaxial nature of the clamp device allows for multi-directional attachment and contouring of reinforcement rod 48 as illustrated in FIG. 5. The clamp device also provides the clinician with the ability to angle the reinforcement system medially and laterally and adjust for misalignment between vertebral segments that are caudal and rostral. This requires more than one degree of freedom otherwise it would be difficult to align the clamp devices. FIG. 6 illustrates a substantially parallel configuration of rods 44 and 48 with an angular rotation with respect to the spinal screw 60. The polyaxial nature of the device allows for housing 88 to be rotatable with respect to spinal screw 60. Housing 88 can be rotated inward toward the vertebrae or centrally to reduce the profile of clamp device 90. Alternatively, housing 88 can be rotated outward with respect to spinal screw 60. Rotation of housing 88 reduces the profile of the overall system and provides more comfort for the patient. Other devices in the art do not include polyaxial clamps and attach to the end of the rods as opposed to between spinal screws as do reinforcement systems of the present invention. As such, in an embodiment, a reinforcement system includes at least two clamp devices positioned between at least two spinal screws in an operative configuration.

After primary rod 44 has been attached to reinforcement rod 48, reinforcement rod 48 can then be locked into place via locking set screw 81. This clamp device allows for attachment to primary rod 44 via base 89. In an embodiment as described above, base 89 has a groove 189 to allow for placement onto an existing spinal rod 44 (i.e. a primary rod) where access to either end of the rod is not possible. Other embodiments are possible where the attachment mechanism is a bore through which the primary rod is inserted prior to being secured on both ends. Base 89 is secured to primary rod 44 by torqueing the internal set screw 84 down on primary rod 44. As shown in the exploded view of FIG. 2, internal setscrew 84 goes through a hole in housing 88 and is threadably engageable therewith. Aperture 87 in of base 89 is threaded to accept internal set screw 84.

The head of internal setscrew 55 is shown with a ball shape but other configurations of head 55 are possible such as a cup shape or a flat top. A cup shape may be used to maximize the surface contact with reinforcement rod 48 to increase the holding force between the locking set screw 81 and reinforcement rod 48. The ball head of the internal setscrew may also have an independent component surface assembled that sits prominent to the ball head of the internal setscrew, so that the internal setscrew can still pivot multi-axially but the contacting separate component makes flat or cupped contact with a reinforcement rod. Similarly, the bottom portion of the locking set screw 82 is shown with a flat surface but may have alternate surface shapes such as a cup shape or a ball shape.

FIGS. 7 and 8 depict alternative embodiments of a reinforcement system. Referring to FIG. 8, reinforcement system 380 comprises a housing 388, a locking set screw 381 that is threadably engaged with the internal threading of the opposing walls of housing 388 to secure a reinforcement rod in place in an operative configuration (similar to the embodiment described above with respect to FIGS. 1 and 2). Reinforcement system 380 comprises a base 356 defining an aperture 360 extending along a first axis. Base 356 further comprises a groove 357 extending along a second axis. Groove 357 is laterally spaced away from aperture 360 of base 356 such the first axis and the second axis do not intersect. Base 356 comprises another internally threaded aperture 354 extending along a third axis generally parallel to the first axis. Internally threaded aperture 354 is substantially aligned with groove 357 such that the second axis and the third axis intersect. Reinforcement system 380 further comprises another locking set screw 353 threadably engageable with internally threaded aperture 354 of base 356. In an operative configuration, locking set screw 353 is threadably engaged with the internal threading of aperture 354 and is secured against a primary rod as similarly described above with respect to reinforcement system 80. Reinforcement system 380 further includes an internal fastener 359 comprising a head 352 having a rounded bottom portion and a shaft 362 connected to the head 352. As with the embodiment described above with respect to FIGS. 1 and 2, the opposing walls of the housing alternatively can be externally threaded to engage a locking set screw with complimentary threading to secure a reinforcement rod in place.

Referring to FIG. 7, a hole 257 can alternatively be used to accept a primary rod. In particular, FIG. 7 depicts a reinforcement system 280 comprises a housing 288, a locking set screw 281 that is threadably engaged with the internal threading of the opposing walls of housing 288 to secure a reinforcement rod in place in an operative configuration (similar to the embodiment described above with respect to FIGS. 1, 2 and 8). Reinforcement system 280 comprises a base 256 defining an aperture 260 extending along a first axis. Base 256 further defines a hole 257 extending along a second axis. Hole 257 is laterally spaced away from aperture 260 of base 256 such the first axis and the second axis do not intersect. Base 256 comprises another internally threaded aperture 254 extending along a third axis generally parallel to the first axis. Internally threaded aperture 254 is substantially aligned with hole 257 such that the second axis and the third axis intersect. Reinforcement system 280 further comprises another locking set screw 253 threadably engageable with internally threaded aperture 254 of base 256. In an operative configuration, locking set screw 253 is threadably engaged with the internal threading of aperture 254 and is secured against a primary rod as similarly described above with respect to reinforcement system 80 and 380. Reinforcement system 280 further includes an internal fastener 259 comprising a head 252 having a rounded bottom portion and a shaft 262 connected to the head 252. As with the embodiment described above with respect to FIGS. 1 and 2, the opposing walls of the housing alternatively can be externally threaded to engage a locking set screw with complimentary threading to secure a reinforcement rod in place.

In the embodiments depicted in FIGS. 7 and 8, the respective shafts 262 and 362 of respective internal fasteners 259 and 359 are integrally attached to respective bases 256 and 356. Alternatively, the fasteners are removable attached to the bases. In a preferred embodiment, the housing is secured to the base but is allowed to rotate polyaxially. The housing can be attached to the base via a screw and ball mechanism such as the internal set screw 84 depicted in FIG. 2. The housing can be oriented such that the reinforcement rod is perpendicular to the primary rod or parallel to the primary rod in an operative configuration.

In any of the embodiments described herein, the clamp device can attach to a single primary rod or one or more primary rods of a spine stabilization construct(s). Further, the groove or hole of the base of the clamp device can be positioned between at least two spinal screws of the spine stabilization construct. The spinal screws are attached to a primary rod of the spine stabilization construct.

Referring back to FIG. 4, a reinforcement system further can include a kit comprising a clamp device and a spinal screw, a spinal rod (such as a primary rod) having a portion receivable by the hole or groove, a reinforcement rod having a portion receivable by the recess, or any suitable combination thereof. The kit can include a plurality of spinal screw rods, spinal rods (such as primary rods), reinforcement rods, or any combination thereof. FIG. 4 illustrates four clamp devices, two bilateral reinforcement rods and two bilateral spinal rods, but a kit can include more or less components.

A reinforcement system can be used in a variety of medical conditions where it is desired to reinforce and further stabilize spinal constructs. A reinforcement system can be used to stabilize a prior construct, such as an existing spine stabilization construct that is already in place. A reinforcement system can attach to such constructs as depicted, for example, in FIG. 4. In such an instance, a reinforcement system is attached to a spinal construct in a follow-up procedure after the spinal construct has been fully implanted. Alternatively, the reinforcement system can be attached to a spinal construct during the same procedure.

Reinforcement systems can be used to connect two separate spine stabilization constructs. For example, if one construct exists in the thoracic spine and second exists in the lumbar spine, a reinforcement system can be used to connect or link these two constructs together and strengthen both constructs as well as the intervening spinal segments.

Regarding an exemplary method of using a reinforcement system with reference to FIG. 4, a spine stabilization construct 139 can be implanted in a patient's spine. An exemplary construct is a traditional bone screw-rod fixation system. A reinforcement system can be used to reinforce a multilevel construct and thus such a system, for example, can reinforce the spine at least three levels of the spinal column. A reinforcement rod can be placed on each side or on one side of construct. To each end portion of this reinforcement rod, two clamp devices can then be affixed. In a preferred embodiment, a parallel reinforcement system is placed posterior to the original spine stabilization construct. In other embodiments, the parallel reinforcement system attaches medial, lateral or oblique to the original spine stabilization construct. The parallel reinforcement system can also be used to bridge two separate spine stabilization constructs or can be used to bridge an existing spine stabilization construct to an adjacent spinal level.

Referring to FIG. 9, in another embodiment, a clamp device 62 is incorporated into a spinal screw 60 to form a setscrew clamp 61, preferably tulip shaped, where primary rod 44 and reinforcement rod 48 extend substantially parallel to each other and both rods are engaged in the same clamp 61. The rods may be touching each other or separated by a saddle, as described in more detail below. Alternatively, referring back to FIGS. 2 and 4, primary rod 44 can be secured with an internal setscrew 84 and the reinforcement rod 48 can be secured with a locking setscrew 81.

As stated above, clamp devices as disclosed herein, allow polyaxial degrees of freedom to simplify rod contouring and insertion. This attachment could be through a “snap-on” or “drop-on” mechanism, or the clamp devices could slide onto the ends of a rod. Certain embodiments provide for a reinforcement system comprising a reinforcement rod and separate clamp devices where the rod can be cut and contoured to the appropriate length intra-operatively, or systems including a reinforcement rod in a pre-cut and/or pre-contoured size with clamp devices pre-attached to the rod. Alternatively, the two clamp devices and reinforcement rod could comprise a single spinal construct available in a variety of lengths.

The clamp devices and parallel reinforcement rods may be integral or separate. In some cases, the reinforcement rods are the same size, but the clamp devices are configured to allow for a variety of parallel reinforcement rod diameters depending on the desired stiffness of the final construct. In certain embodiments, the rods that the parallel reinforcement rods will attach to have different diameters.

Regarding a kit comprising clamp devices, the clamp devices can be different sizes and therefore a clinician can use different clamp devices, one on the caudal side of a parallel reinforcement system and one on the rostral side of the parallel reinforcement system.

Regarding other aspects, the diameter of a reinforcement rod is preferably between about 2 millimeters (mm) and 10 mm. In a preferred embodiment, the reinforcement rod is about 5.5 mm but can be larger or smaller in diameter as needed. Reinforcement rods can be made in lengths that range from 1 segment (30mm) to 10+ segments. Reinforcement rods can be either pre-contoured or straight, allowing the surgeon to perform contouring in situ via the use of bending instruments. Spinal rods, such as primary rods for example, can range from 3.0 mm to 6.35 mm in diameter, but they can be other sizes as well. The spinal rods, primary rods, and reinforcement rods can be cylindrical, rectangular, flat on one side or have other suitable configurations. The rods an also be grooved or have a spiral configuration. Common rod materials are titanium, stainless steel, cobalt chrome, and PEEK.

Referring to FIG. 15-20, the present invention provides other embodiments of a clamp device and a reinforcement system comprising a clamp device and at least one reinforcement rod. Any suitable features described with respect to these clamp devices and reinforcement systems can be incorporated in the other clamp devices disclosed herein. Referring to FIG. 15, in an embodiment, a reinforcement system 110 comprises a clamp device 190 comprising a housing 12, which is preferably tulip-shaped. Housing 12 comprises first and second opposing upper walls 14a and 14b separated by a bore 16 extending along a first axis. With reference to FIGS. 17 and 18, bore 16 is larger in diameter than reinforcement rod 148 (described in more detail below) to allow for variability in the angle in which reinforcement rod 148 engages bore 16. Primary rod 144 (described in more detail below) may not be parallel with the spinal rod on the opposite side of the original construct. The angular variability of reinforcement rod 148 accommodates for a reinforcement rod 148 that is skewed with respect to housing 12. Housing 12 further comprises first and second opposing lower walls 18a and 18b generally perpendicular to opposing upper walls 14a and 14b and separated by a recess 20 extending along a second axis generally perpendicular to the first axis. At least one of the opposing lower walls 14a and 14b has a top surface 22 defining a groove 24.

Reinforcement system 110 further comprises a saddle 26 having flexure properties and sized to be received in housing 12. Saddle 26 has first and second opposing lower walls 28a and 28b separated by a recess 30 extending along the second axis. As seen in FIG. 18, each of the first and second opposing lower walls 28a and 28b of saddle 26 has an interior face 34a and 34b and an exterior face 46a and 46b. In an operative configuration, interior faces 34a and 34b are positioned towards primary rod 144 (described in more detail below) and exterior faces 46a and 46b are positioned toward first opposing lower wall 18a and second opposing lower wall 18b, respectively, of housing 12. Preferably, each of the interior faces of saddle 26 has a substantially arcuate shape as seen in FIG. 18. Such an arcuate shape can help secure primary rod 144 in recess 30 of saddle 26. Referring to FIG. 17, in certain embodiments, reinforcement system 110 further comprising a slot 42 extending through the opposing upper walls 14a and 14b of housing 12 and a pin (not shown) configured to engage slot 42. The pin can be located above saddle 26 and can be secured by pressing into housing 12 after saddle 26 is inserted during manufacturing to secure saddle 26 in housing 12. Other methods of securing saddle 26 in housing 12 are also possible to prevent saddle 26 from falling out of housing 12 in an operative configuration.

Reinforcement system 110 further comprises a fastener 32 configured to be received by the opposing upper walls 14a and 14b of housing 12. In particular, referring to FIG. 18, housing 12 can comprise another bore 34 extending along a third axis generally perpendicular to the first and second axes that is configured to receive fastener 32. The first and second opposing upper walls 14a and 14b of housing 12 each have an inner face 36a and 36b and an outer face 38a and 38b. The inner faces can be at least partially threaded. In such embodiments, the fastener of reinforcement system 110 can be a locking screw with exterior threads 40 complimentary to the at least partially threaded inner faces 36a and 36b of the housing's opposing upper walls 14a and 14b so that fastener 32 threadably engages the inner faces of the opposing upper walls as seen in FIG. 18. Fastener 32 secures secondary rod 48 against groove 24 and also secures saddle 26 onto rod 44.

Referring to FIGS. 16 and 17, in certain embodiments, a reinforcement system 110 includes orthopedic rods 148. Such a reinforcement system 110 can further includes a primary rod 144, configured to be secured by recess 30 of saddle 26 via a snap fit, example. In such embodiments, the substantially arcuate shape of inner faces 34a and 34b of saddle 26 assist in capturing and retaining primary rod 144 in an operative configuration. In embodiments, reinforcement system 110 can further comprise a reinforcement rod 148, which can be a spinal rod, configured to be received by groove 24 of housing 12. To that end, in certain embodiments, saddle 26 comprises a top face 50 from which opposing lower walls 28a and 28b downwardly extend. Top face 50 of the saddle 26 and opposing upper walls 14a and 14b of housing 12 effectively define a receptacle configured to receive a secondary rod 48. Groove 24 allows reinforcement rod 148 to be “dropped into” housing 12.

Referring to FIG. 19, in another embodiment, the present invention provides a reinforcement system 210 comprising a housing 100 comprising first and second opposing upper walls 112a and 112b separated by a bore 114 extending along a first axis. Housing 100 further comprises first and second opposing lower walls 118a and 118b substantially perpendicular to opposing upper walls 112a and 112b and separated by a recess 120 extending along a second axis substantially perpendicular to the first axis. At least one of the walls 112a or 112b defines an elongate flexure cut 116. Preferably, flexure cut 116 is generally perpendicular to the first and second axes. At least one of the opposing lower walls 118a or 118b has a top surface 122 defining a groove 124. First and second opposing lower walls 118a and 118b have an inner face 128a and 128b respectively that each has a substantially arcuate shape. This arcuate shape assists in capturing and retaining rod 132, such as a primary rod (shown in FIG. 20) in an operative configuration.

Referring to FIG. 20, system 210 further comprises fastener 126 configured to be received by opposing upper walls 112a and 112b of housing 100. In particular, housing 100 comprises another bore extending along a third axis, substantially perpendicular to first and second axes, that is configured to receive fastener 126. Each of the first and second opposing upper walls 112a and 112b has an inner face (inner face 134b of second upper wall 112b shown in FIG. 19) that is preferably at least partially threaded. In those embodiments, fastener 126 is a locking screw with exterior threads complimentary to the at least partially threaded inner faces of the housing's opposing upper walls.

In another embodiment, a reinforcement system further includes orthopedic rods. Referring to FIG. 20, reinforcement system 210 can further comprise a rod 132, such as a primary rod, configured to be secured by recess 120 via a snap fit. In particular, flexure cut 116 allows housing 100 to snap onto rod 132. System 92 can further comprise an additional rod 138, such as a reinforcement rod, configured to be received by groove 124 of housing 100.

Rod 138 can be dropped into the u-shaped yoke or groove 124 of housing 100. When fastener 126 is tightened, rod 138 is seated into housing 100 and housing 100 clamps onto rod 132. Alternatively, the housing may have a hole or slot instead of a yoke so that rod 138 is inserted into the slot or hole instead of the rod being dropped into the yoke. The groove 24 in housing 12 of reinforcement system 110 and recess 122 in housing 100 of reinforcement system 210 described above provides for a “drop-in” slot for ease of insertion. The snap fit design of the saddle 26 or the housing 100 requires tightening of only one screw (i.e. screw 32 or screw 126).

FIG. 21 shows another embodiment of a reinforcement system 310 in which saddle 26 as described above and illustrated in FIGS. 15 and 16, is not used and reinforcement rod 348 and primary rod 44 are in direct contact with each other.

As stated above, mechanical fixation of a spinal level requires attachment of a rigid fixator to portions of the spinal elements (vertebral body, pedicle, lateral mass, transverse process, facet joint, lamina, spinous process). The most common forms of rigid screw-rod based fixation used in clinical practice include attachment of a bone screw to either the pedicle or lateral mass of a given spinal level. There is a high incidence of adjacent segment disease where after fusing a spinal segment or multiple segments. The adjacent segment becomes degenerated and additional surgery is needed. This usually entails fusing the next segment with the same traditional methods used above. A reinforcement system that is a rigid spinous process plating system, depicted in FIGS. 10, 11 and 13, on a spine offers a minimally invasive way to fuse the next vertebral segment during a reoperation or initial placement of a spinal screw and rod system in the case that a previous laminectomy has been performed. The rigid spinous process plating system connects to the primary rods of an existing spinal screw construct and to the spinous process 180 of the adjacent segment. Examples of reinforcement systems 400 and 500 that are rigid spinous process plating systems are depicted in FIGS. 10 and 11. FIG. 12 depicts an existing spine stabilization construct 159 and FIG. 13 illustrates a reinforcement system attached to the existing spine stabilization construct.

As such, an embodiment of the present invention provides a spinal augmentation fixation system. A rigid spinous process plating system is a one level fusion system for fusing adjacent to an existing fusion without placing additional spinal screws, in the setting of a prior laminectomy.

FIGS. 10 and 11 show preferred embodiments of a respective system 400 and 500 Reinforcement systems 400 and 500 comprises at least two clamp devices 480/580, each comprising a housing comprising an aperture extending along a first axis and a bore extending along a second axis substantially perpendicular to the first axis. The bore is configured to accept a portion of a reinforcement rod 448/558. The housing further comprises a groove extending along a third axis substantially perpendicular to the first and second axes. The groove is configured to accept a portion of a primary rod 444/544. Clamp device further includes a set locking screw threadably engageable with the aperture of the housing. Reinforcement system 400/500 further includes opposing rigid plates 491/591 each having an upper portion and a lower portion. The lower portion connects to a reinforcement rod 448/548 and the upper portion connects to a spinous process in an operative configuration. Reinforcement system 400/500 further includes a reinforcement rod 448/548 connected to the lower portions of the opposing rigid plates 491/591 and the at least two clamp devices 480/580.

In particular, systems 400 and 500 include a clamp device 480 and 580, respectively, which are preferably tulip-shaped. These clamp devices attach to a primary rod. A primary rod 444 of a spine stabilization construct 159 is illustrated in FIG. 12. Systems 400 and 500 also include a reinforcement rod 448 and 548, respectively, which attach to clamp devices 480 and 580, respectively, and creates a cross-connection between the first primary and second primary rods. Although also applicable to system 500, FIG. 13 illustrates such a cross-connection between first primary and second primary rods 444a and 444b, respectively, where reinforcement rod 448 is substantially perpendicular to both the first primary and second primary rods. Given misalignment of vertebral segments and facet joints, reinforcement rod 448 may be skew with respect to the first and second primary rods 444a and 444b.

The reinforcement rods can be separate from the clamp devices and can be cut to the appropriate length intra-operatively, or can be pre-cut and/or pre-contoured size to mate with the clamp devices. Alternatively, a clamp device can be pre-attached to a reinforcement rod. Alternatively, two clamp devices and a reinforcement rod can comprise a single spinal construct available in a variety of lengths.

A clamp device can be, for example, any of the clamp devices disclosed herein. With reference to FIGS. 1 and 14, in a preferred embodiment, a clamp device 90 attaches to an existing primary rod 444a via groove 189. As described above, housing 88 has a recess substantially perpendicular to primary rod 444 and groove 189 for accepting reinforcement rod 448. Groove 189 is sized to allow for misalignment of reinforcement rod 448 due to the first primary rod 444a and the second primary rod 444b not being parallel. Housing 88 is attached to primary rod 444 via set screw 84. Reinforcement rod 448 is then locked into place via locking set screw 81, which locks both the primary rod 444 and the secondary rod 448 onto housing 88 simultaneously. In a preferred embodiment, reinforcement rod 448 is also attached to a variety of different features or devices. Referring to FIGS. 10 and 11, one such device is a rigid plate 491 and 591, respectively, that attaches to the adjacent spinous process via a rigid plate pin 492 and or a plurality of teeth 592, respectively. In the embodiment depicted in FIG. 10, pin 492 is secured to plate 491 via a fastener 493.

Rigid plate 491/591 is used to connect a spinous process 180 to a reinforcement rod 448 as illustrated in FIG. 13. Rigid plate 491/591 may be made of materials such as but not limited to titanium, cobalt chrome, PEEK, CFRP (Carbon Fiber Reinforced PEEK), high durometer polyurethane, or other rigid materials. The plates act to stabilize the adjacent segment of an existing construct and may be used in conjunction with an interbody fusion device placed in the corresponding vertebral disc space, or as a stand-alone device for spinal fusion.

Referring to FIGS. 10 and 13, during operation, a hole is cut into spinous process 180 that is sized to accept a rigid plate pin 492. The configuration of the rigid plate 491 may include a hole that is larger than the rigid plate pin 492 that goes through it. In an embodiment, rigid plate 491 has a slot instead of a hole connecting to pin 492 going through spinous processes 180. A slot has multiple benefits. First, a slot can adjust for multiple distances between spinous processes. The distance between spinous processes can vary dramatically from patient to patient. Creating devices that are specifically customized for each patient is not practical. Therefore, creating a slotted device allows for a minimal number of device sizes and yet allows some customization for each patient.

A slot can also be advantageous for insertion of an elastic material. The elastic material can be made of but is not limited to silicone, polyurethane, Elasthane, Biothane, and other blends of polyurethane and silicone. A spinous process is susceptible to fracture and putting an elastic material in the hole or around the connection site to a spinous process can reduce the stress on the spinous process and reduce the risk of fracture. Additionally, other portions of the rigid plate can be made of an elastic material to allow for reducing stress on the spinous process. Also, rigid plate pin 492 that goes through the hole in spinous process 180 may be coated with a porous titanium or other bone stimulating materials to help create bone ingrowth.

Other embodiments of a reinforcement system are also provided. The slots on rigid plate may be on one or both sides. In addition, there may be a different combination of slots and holes for each of the rigid plates of the construct. This can allow for multiple different sizes of spinous process distances to be covered with one device.

In addition, the spinous process connection member of a rigid plate may be either integrated directly into the rigid or semi-rigid plate, or may come as a separate connector. Referring to FIG. 11, as a way of attaching to the adjacent spinous process, “teeth” 592 can also be located on the inner side of rigid plate 591 in order to allow adherence to the cortical surface of a spinous process 180.

The systems provide connection of a rigid plate from a spinal screw-rod construct 159 to a spinous process 180 in the setting of a decompressive laminectomy; minimally invasive fusion; use in combination with an interbody fusion, or as a stand-alone fusion device; and/or a bar that can accept attachments such as a rigid fusion to the next level without the need for adjacent level spinal screw fixation. The method described above details two rigid plates 491/591 on either side of the spinous process. Other methods may be used where only one rigid plate 491/591 is used on either side of the spinous process. In yet another embodiment, a single rigid plate is in line with the spinous process and connected to the spinous process via forked connector, lasso, or other connection method. Although a rigid plate has been detailed other potential geometries could be contemplated such as a rod or other rigid shapes.

For certain spinal fixation or dynamic stabilization devices, it may be necessary to punch a hole in the spinous process. The present invention provides embodiments of a system and method for making a hole in the spinous process as described in Provisional Application No. 61/787,763, pages 14-16 and also illustrated in FIGS. 24 and 25, this application being incorporated by reference herein. The spinous process target tool is used to locate the portion of the spinous process that the hole is to be made. In a preferred embodiment, the majority of the tool is made of a material that is not radiopaque such as but not limited to ultem, polycarbonate, delron, PEEK, polypropylene and other plastic materials. In other embodiments, the entire tool or the majority of the tool can be made of a radiopaque material such as but not limited to stainless steel. The device has a proximal end, a body, and distal end. The distal end contains a spinous process target hole and is configured to attach to the spinous process. The distal end has a radiopaque portion that creates a target for the hole in the spinous process. This hole can be seen under fluoroscopy. In a preferred embodiment, a radiopaque material is embedded in a non-radiopaque material so as to minimize the amount of the spinous process that is obscured when taking a fluoroscopy image with the spinous process target tool on the spinous process. In another embodiment, substantially all of the distal end may be radiopaque. In a preferred embodiment, the distal end also contains a shelf. As the spinous process tool is advanced on the spinous process, the shelf stops the advancement of the tool when it contacts the most posterior portion of the spinous process. This ensures that the spinous process target tool hole is a certain distance from the most posterior portion of the spinous process. The shelf may be perpendicular to the longitudinal axis of the tool or it may be angled so the tool may be applied to the spinous process at an angle. The distal end of the distal portion of the tool can also comprise a lead in to help with applying the tool to the spinous process by applying a force in the anterior direction as the tool gets close to the posterior portion of the spinous process.

The body of the spinous process tool may contain a trough, the trough being configured to accept a spinous process punch tool. The trough extends distally to the distal end of the tool and more specifically to the target hole in the distal end of the tool. In one embodiment, the tool has a trough entry slot on the side to make it easier for the punch tool to enter the trough. The trough may also extend proximally to the proximal end.

The proximal end of the tool remains outside the patient's body. In a preferred embodiment, the tool's natural state is closed. The proximal end of the tool contains a living hinge so that when a force is applied at the distal end, the tool can be opened. The living hinge provides enough force for the distal end of the tool to clamp down on the spinous process keeping it in place without assistance. This is so a fluoroscopic image can be taken without a physician's hands being in the fluoroscopy area or any other tools needed. Other embodiments include where the natural state is closed and a pivot point and a spring exists in either the proximal end or the body of the tool such that when a force is applied at the proximal end of the tool the distal end opens. In another embodiment, the natural position of the tool is open and when a closing force is applied at the proximal end the distal end closes and a ratcheting device is contained in the proximal end or the body to keep the device closed.

In certain embodiments, a method of creating a hole in a spinous process involves identifying a spinal segment in which a hole is needed; applying a spinous process target device; while applying the spinal process target tool, advancing a tool until a shelf hits the most posterior portion of the spinal process (optional); taking a fluoroscopy image to confirm the spinous process target is in the correct place; engaging the spinous process punch into a trough of the spinous process target (optional); sliding the spinous process punch into the spinous process target hole; create a hole in the spinous process with the spinous process punch; removing the spinous process punch; reimage to confirm the hole is in the correct place (optional).

Embodiments of the present invention provide a tool for identifying a portion of the spinous process in which to create a hold; a tool with a trough for engaging a punch tool; a tool with a shelf for determining the depth at which to create a hole in the spinous process; a method for creating a hole in the spinous process; and a tool with a lead in for advancing onto the spinous process.

The foregoing description and examples have been set forth merely to illustrate the invention and are not intended as being limiting. Each of the disclosed aspects and embodiments of the present invention may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. A reinforcement system for a spine stabilization construct, the reinforcement system comprising in an operative configuration:
at least two clamp devices, each comprising:
a polyaxial fastener defining a recess securing a reinforcement rod therein; and
a base positioned below and engaged with the polyaxial fastener, the base comprising a groove or hole securing a primary rod therein of the spine stabilization construct; and
a reinforcement rod secured in the recesses of the polyaxial fasteners of the at least two clamp devices and connected to the primary rod via the at least two clamp devices.

2. The reinforcement system for a spine stabilization construct of claim 1, wherein each of the at least two clamp devices comprise:
the polyaxial fastener system comprising:
a housing having opposing walls that are at least partially threaded and separated by the recess;
an internal fastener comprising a head having a rounded bottom portion and a shaft connected to the head; and
a locking set screw threadably engaged with the at least partially threaded opposing walls of the housing; and
the base comprising:
an aperture extending along a first axis and engaged with the shaft of the internal fastener; and
the groove or the hole extending along a second axis substantially perpendicular to the first axis.

3. The reinforcement system of claim 1, wherein the groove or hole is positioned between at least two spinal screws of the spine stabilization construct, the spinal screws attached to a primary rod of the spine stabilization construct

4. The reinforcement system of claim 1, wherein the primary rod is a single primary rod.

5. The reinforcement system of claim 1, wherein the primary rod is two or more primary rods.

6. The reinforcement system of claim 2, wherein the groove and the aperture of the base are substantially aligned such that the first axis and second axis intersect.

7. The reinforcement system of claim 2, wherein the internal fastener is a set screw, the shaft is externally threaded, the aperture is internally threaded, and the aperture is threadably engaged with the externally threaded shaft of the set screw.

8. The reinforcement system of claim 2, wherein the groove of the base is laterally spaced away from the aperture of the base such that the first axis and the second axis do not intersect.

9. The reinforcement system of claim 6, wherein the base comprises another internally threaded aperture extending along a third axis generally parallel to the first axis and the another internally threaded aperture is substantially aligned with the groove such that the second axis and the third axis intersect.

10. The reinforcement system of claim 9, further comprising another locking set screw threadably engaged with the another internally threaded aperture of the base.

11. The reinforcement system of claim 2, wherein the shaft of the fastener is integrally engaged with the base.

12. The reinforcement system of clam 1, wherein the base comprises the hole.

13. The reinforcement system of claim 12, wherein the hole is laterally spaced away from the aperture of the base such that the first axis and second axis do not intersect.

14. The reinforcement system of claim 13, wherein the base comprises another internally threaded aperture extending along a third axis substantially parallel to the first axis and substantially aligned with the hole such that the second axis and the third axis intersect.

15. The reinforcement system of claim 14, further comprising another locking set screw threadably engaged with the another internally threaded aperture of the base.

16. The reinforcement system of claim 12, wherein the shaft of the fastener is integrally engaged with the base.

17. A kit comprising the reinforcement system of claim 1 and further comprising a spinal screw, a primary rod, or a combination thereof.

18. A kit comprising the reinforcement system of claim 1 and further comprising a plurality of spinal screws, a plurality of primary rods, a plurality of reinforcement rods, or any suitable combination thereof.

19. The reinforcement system of claim 2, wherein the clamp device is incorporated with a spinal screw.

20. A reinforcement system for a spine stabilization construct, the reinforcement system comprising in an operative configuration:
at least two clamp devices each comprising:
a housing comprising:
an aperture extending along a first axis; a bore extending along a second axis substantially perpendicular to the first axis, the bore securing a reinforcement rod therein;
a groove extending along a third axis substantially perpendicular to the first and second axes, the groove securing a primary rod therein;
a set locking screw threadably engaged with the aperture of the housing;
opposing rigid plates each having an upper portion and a lower portion, the lower portions connected to a reinforcement rod and the upper portion connected to a spinous process; and
a reinforcement rod connected to the lower portions of the opposing rigid plates and the at least two clamp devices.

21. The reinforcement system of claim 20, wherein the upper portion of at least one of the opposing plates comprises a pin.

22. The reinforcement system of claim 20, wherein the upper portion of at least one of the opposing plates comprises teeth.
